(19) United States
(12) Patent Application Publication
(10) Pub. No.: US 2004/0038288 A1
(43) Pub. Date: Feb. 26, 2004

(54) Human protein C Polypeptide

(76) Inventors: Lihua Huang, Carmel, IN (US); Ralph M. Riggin, Carmel, IN (US)

Correspondence Address: ELI LILLY AND COMPANY, PATENT DIVISION, P.O. BOX 6288, INDIANAPOLIS, IN, 46206-6288, US

(21) Appl. No.: 10/670,628

(22) Filed: Sep. 25, 2003

Related U.S. Application Data
(63) Continuation of application No. 09/763,153, filed on Feb. 20, 2001, now abandoned.
Application No. 09/763,153 is a 371 of international application No. PCT/US99/11969, filed on Jun. 1, 1999.
(60) Provisional application No. 60/087,585, filed on Jun. 1, 1998.

(30) Foreign Application Priority Data
Dec. 9, 1999 (WO) 99/63070

(51) Int. Cl. C12Q 1/68; C07H 21/04; A61K 38/48; C12N 9/64

(52) U.S. Cl. 435/6; 435/69.1; 435/320.1; 435/226; 435/325; 424/94.64; 536/23.2

(57) ABSTRACT

An isolated human protein C polypeptide with a truncated heavy chain is described. This isolated polypeptide retains the biological activity of the wild-type human protein C. This polypeptide will be useful in the treatment of vascular occlusive disorders, hypercoagulable states, thrombotic disorders and disease states predisposing to thrombosis.

The present invention is in the field of human medicine. Most specifically, the invention relates to an isolated human protein C polypeptide having a truncated heavy chain, methods of using this human protein C polypeptide, and pharmaceutical compositions of this human protein C polypeptide.

Protein C is a vitamin K dependent serine protease and naturally occurring anticoagulant that plays a role in the regulation of vascular homeostasis by inactivating Factors Va and VIIIa in the coagulation cascade. Human protein C is made primarily in the liver as a single polypeptide of 461 amino acids. This precursor molecule undergoes multiple post-translational modifications including 1) cleavage of a 42 amino acid signal sequence; 2) proteolytic removal from the one chain zymogen of the lysine residue at position 156 and the arginine residue at position 157 to make the 2-chain form of the molecule, (i.e., a light chain of 155 amino acid residues attached through a disulfide bridge to the serine protease-containing heavy chain of 262 amino acid residues); 3) vitamin K-dependent carboxylation of nine glutamic acid residues clustered in the first 42 amino acids of the light chain, resulting in 9 gamma-carboxyglutamic acid residues; and 4) carbohydrate attachment at four sites (one in the light chain and three in the heavy chain). Finally, the circulating 2-chain zymogen is activated by the action of the thrombin/thrombomodulin complex which cleaves the activation peptide (residues 158 through 169) of the circulating zymogen producing activated protein C (aPC).

In conjunction with other proteins, protein C functions as perhaps the most important down-regulator of blood coagulation factors that promote thrombosis. Thus, the protein C enzyme system represents a major physiological mechanism of anticoagulation.

The critical role of protein C in controlling hemostasis is exemplified by the increased rate of thrombosis in heterozygous deficiency, protein C resistance (e.g., due to the common Factor V Leiden mutation) and the fatal outcome of untreated homozygous protein C deficiency. Human activated protein C, both plasma-derived and recombinant, has been shown to be an effective and safe antithrombotic agent in a variety of animal models of both venous and arterial thrombosis. Protein C in recent clinical studies has been shown to be effective in human thrombotic diseases including the treatment of protein C deficiencies and microvascular thrombosis, such as disseminated intravascular coagulation associated with sepsis.

Unfortunately, during activation of protein C, the C-terminal of the heavy chain is cleaved which has th potential to change the protein's structure, which in turn may lead to a less elegant pharmaceutical preparation. Applicants have discovered that this truncated form of aPC is biologically active. The present invention therefore provides an isolated aPC polypeptide with a truncated heavy chain, a method to preferentially prepare this polypeptide, and its use as a medicament.

The present invention provides anisolated human protein C polypeptide comprising: a light chain and a truncated heavy chain wherein said polypeptide is SEQ ID NO: 1.

The present invention further provides a recombinant DNA molecule encoding the isolated human protein C polypeptide with a truncated heavy chain, wherein said DNA molecule is SEQ ID NO: 2.

The present invention further provides a method of treating a thrombotic disease in a patient in need thereof, which comprises, administering to said patient a pharmaceutically effective amount of an isolated human protein C polypeptide with a truncated heavy chain.

Methods and aspects of producing the isolated human protein C polypeptide with a truncated heavy chain are also an aspect of this invention.

For purposes of the present invention, as disclosed and claimed herein, the following terms are as defined below.

aPC or activated protein C whether recombinant or plasma derived—aPC includes and is preferably human protein Calthough aPC may also include other species or derivatives having protein C proteolytic, amidolytic, esterolytic, and biological (anticoagulant or pro-fibrinolytic) activities. Examples of protein C derivatives are described in U.S. Pat. No. 5,453,373, and U.S. Pat. No. 5,516,650, the entire teachings of which are hereby included by reference.

APTT—activated partial thromboplastin time.

HPC—human protein C zymogen.

r-hPC—recombinant human protein C zymogen, produced in prokaryotic cells, eukaryotic cells or transgenic animals.

r-aPC—recombinant human activated protein C produced by activating r-hPC in vitro or by direct secretion of the activated form of protein C from procaryotic cells, eukaryotic cells, or transgenic animals [WO97/20043] including, for example, secretion from human kidney 293 cells as a zymogen then purified and activated by techniques well known to the skilled artisan demonstrated in U.S. Pat. No. 4,981,952, and, the entire teachings of which are herein incorporated by reference.

Zymogen—refers to secreted, inactive forms, whether one chain or two chains of protein C.

Truncated heavy chain—refers to the heavy chain of protein C having its four C-terminal amino acids cleaved. For human activated protein C, the truncated heavy chain contains amino acid residues 170-415 as indicated in SEQ ID No: 1.

Light chain—refers to the light chain of protein C. For human activated protein C, the light chain contains amino acid residues 1-155 or polypeptides having one or more amino acids deleted from th C-terminus.

Thrombotic disorder—a disorder relating to, or affected with the formation or presence of a blood clot within a blood vessel. Thrombotic disorders include, but are not limited to, stroke, myocardial infarction, unstable angina, abrupt closure following angioplasty or stent placement, and thrombosis as a result of peripheral vascular surgery.

Vascular occlusive disorders and hypercoagulable states: disorders including but not limited to sepsis, disseminated intravascular coagulation, purpura fulminans, major trauma, major surgery, burns, adult respiratory distress syndrome, transplantations, deep vein thrombosis, heparin-induced thrombocytopenia, sickle cell disease, thalassemia, viral hemorrhagic fever, thrombotic thrombocytopenic purpura, and hemolytic uremic syndrome

Pharmaceutical formulation—a formulation or solution that is appropriate to be given as a therapeutic agent.

Pharmaceutically effective amount as used herein, represents an amount of a compound of the invention that is capable of inhibiting a thrombotic disorder in mammals. The particular dose of the compound administered according to this invention will, of course, be determined by the particular circumstances surrounding the case, including the compound administered, the particular condition being treated, and similar considerations.

The structure of HPC is rather complex due to the number of post-translational modifications. The HPC structure consists of a light chain (residues 1-155) and a heavy chain (residues 158-419). The HPC molecule is originally expressed as a 419 amino acid polypeptide, but prior to secretion from the cell, most of the protein is converted to the heterodimer form by removal of the Lys-Arg dipeptide at positions 156-157.

Recombinant human protein C (r-hPC) is analogous to HPC in its structure and complexity. During the conversion of r-hPC to r-aPC. thrombin selectively cleaves the activation dodecapeptide (residues 158-169). However, applicants have discovered conditions where a tetrapeptide (residues 416-419) may also cleaved from the C-terminus of the heavy chain resulting in the formation of des 416-419 aPC polypeptide applicants have further discovered that this form of aPC is biologically active (see Example 1, Table 1), leading to its use as a therapeutic alone or in combination with native aPC. The present invention therefore provides isolated des (416-419) aPC, a method to preferentially prepare des (416-419) aPC, and its use as a medicament

The invention also provides DNA compounds for use in making the protein C having a truncated heavy chain. These DNA compounds comprise the coding sequence for the light chain of human protein C positioned immediately adjacent to, downstream of, and in translational reading frame with the prepropeptide sequence of wild-type zymogen protein C. The DNA sequences also encode the Lys-Arg dipeptide which is processed during maturation of the protein C molecule, the activation peptide and the truncated heavy chain of the protein C molecule.

Those skilled in the art will recognize that, due to the degeneracy of the genetic code, a variety of DNA compounds can encode the activated protein C polypeptide described above. U.S. Pat. No. 4,775,624, the entire teaching of which is herein incorporated by reference, discloses and claims the DNA sequence encoding the wild-type form of the human protein C molecule. In that the skilled artisan could readily determine which changes in the DNA sequences might be used to construct the other DNA sequences which could encode the exact polypeptide as disclosed herein, the invention however is not limited to the specific DNA sequences. Consequently, the construction described below for the preferred DNA compound, vectors and transformants of the invention are merely illustrative and do not limit the scope of the invention.

The DNA compound of the present invention may be prepared by site-directed mutagenesis of the human protein C gene. The cultures are obtained and the plasmids are isolated using conventional techniques, and then may be directly transfected into eukaryotic host cells for the production of protein C with a truncated heavy chain. It is preferable to transfect the plasmids into host cells which express the adenovirus EIA immediate-early gene product, in that the BK enhancer found in the GBMT transcription control unit functions to enhance expression most efficiently in the presence of BEA. The GBMT transcription control unit is more fully described in U.S. Pat. No. 5,573,938 and in European Patent Application Serial No. 91301451.0, the entire teachings of which are herein incorporated by, reference. Skilled artisans realize that a number of host cells express, or can be made to express, an immediate early gene product of a large DNA virus. The most preferred cell line for expression of the human protein C derivatives of the present invention is the human kidney 293 cell line which is disclosed in U.S. Pat. No. 4,992,373, the entire teaching of which is herein incorporated by reference. After expression in the cell line, the derivatives are purified from the cell culture supernatant using the procedure in U.S. Pat. No. 4,981,952, the entire teaching of which is herein incorporated by reference.

The DNA sequence of the invention can be synthesized chemically, or by combining restriction fragments, or by a combination of techniques known in the art. DNA synthesizing machines are available and can be used to construct the DNA compounds of the present invention.

The illustrative vectors of the invention comprise the GBMT transcription unit positioned to stimulate transcription of the coding sequences by the adenovirus late promoter. Those skilled in the art recognize that a great number of eukaryotic promoters, enhancers, and expression vectors are known in the art and can be used to express the DNA sequences to produce the protein C derivatives of the present invention. Those skilled in the art also recognize that a eukaryotic expression vector can function without an enhancer element. The key aspect of the present invention resides in the novel DNA sequences and corresponding aPC with a truncated heavy chain made from those sequences.

Alternatively, the activated protein C polypeptide described herein may be prepared by reacting activated protein C with thrombin to cleave the tetrapeptide (residues 416-419) from the C-terminus of the heavy chain. The additional cleavage is obtain d by exposing aPC to thrombin for an extended period, generally, 10 minutes to 3 to 5 hours under conditions appreciated in the art. aPC polypeptides prepared by treating r-aPC with thrombin or by direct expression from eukaryotic cells have similar activity as aPC. Therefore, aPC having a truncated heavy chain will be effective in the treatment of human thrombotic diseases including replacement therapy in the treatment of protein C deficiencies, vascular occlusive disorders and hypercoagulable states including: sepsis, disseminated intravascular coagulation, purpura fulminans, major trauma, major surgery, burns, adult respiratory distress syndrome, transplantations, deep vein thrombosis, heparin-induced thrombocytopenia, sickle cell disease, thalassemia, viral hemorrhagic fever, thrombotic thrombocytopenic purpura, and hemolytic uremic syndrome as well as thrombotic disorders and disease states predisposing to thrombosis, such as, myocardial infarction and stroke, by administering an isolated human protein C polypeptide having a truncated heavy chain.

Another embodiment of the present invention is a method of treating thrombotic disorders which comprises: administering to a patient in need thereof a pharmaceutically effective amount of an isolated human protein C polypeptide having a truncated heavy chain in combination with an antiplatelet agent.

Another embodiment of the present invention is a method of treating sepsis comprising the administration to a patient in need thereof a pharmaceutically effective amount of an isolated human protein C polypeptide having a truncated heavy chain in combination with bacterial perm ability increasing protein.

An isolated human protein C polypeptide having a truncated heavy chain may be formulated in a manner analogous to aPC with a pharmaceutically acceptable diluent. Preferably, including a sugar such as sucrose, salt, and a citrate buffer. Preferably, aPC derivatives are prepared at a pH of 5.5 to 6.5. Generally, pharmaceutical doses of aPC derivatives described herein will be analogous to those of native aPC, preferably 0.01 mg/kg/hr to 0.05 mg/kg/hr.

The following preparations and examples are for illustrative purposes only. One with skill in the art realizes that there are additional methods to prepare and activate recombinant protein C.

### Preparation 1

### Preparation of Human Protein C

Recombinant human protein C (r-HPC) is produced in Human Kidney 293 cells by techniques well known to the skilled artisan such as those set forth in U.S. Pat. No. 4,981,952, the entire teaching of which is herein incorporated by reference. The gene encoding human protein C is disclosed and claimed in, U.S. Pat. No. 4,775,624, the entire teaching of which is incorporated herein by reference. The plasmid used to express human protein C in 293 cells is plasmid pLPC which is disclosed in U.S. Pat. No. 4,992,373 and U.S. Pat. No. 5,661,002, the entire teachings of which are incorporated herein by reference. The construction of plasmid pLPC is also described in European Patent Publication No. 0 445 939, and in Grinnell, et al., 1987, Bio/Technology 5:1189-192, th teachings of which are also incorporated herein by reference. Briefly, the plasmid is transfected into 293 cells, then stable transformants are identified, subcultured and grown in serum-free media. After fermentation, cell-free medium is obtained by microfiltration.

The human protein C is separated from the culture fluid y an adaptation of the techniques in U.S. Pat. No. 4,981,952, the entire teaching of which is herein incorporated by reference. The clarified medium is made 4 mM in EDTA before it is absorbed to an anion exchange resin (Fast-Flow Q, Pharmacia). After washing with 4 column volumes of 20 mM Tris, 200 mM NaCl, pH 7.4 and 2 column volumes of 20 mM Tris, 150 mM NaCl, pH 7.4, the bound recombinant human protein C zymogen is eluted with 20 mM Tris, 150 mM NaCl, 10 mM CaCl₂, pH 7.4. The eluted protein is greater than 95% pure after elution as judged by SDS-polyacrylamide gel electrophoresis.

Further purification of the protein is accomplished by making the protein 3 M in NaCl followed by adsorption to a hydrophobic interaction resin (Toyopearl Phenyl 650M, TosoHaas) equilibrated in 20 mM Tris, 3 M NaCl, 10 mm CaCl₃₁ pH 7.4. After washing with 2 column volumes of equilibration buffer without CaCl₂, the recombinant human protein C is eluted with 20 mM Tris, pH 7.4. The eluted protein is prepared for activation by removal of residual calcium. The recombinant human protein C is passed over a metal affinity column (Chelex-100, Bio-Rad) to remove calcium and again bound to an anion exchanger (Fast Flow Q, Pharmacia). Both of these columns are arranged in series and equilibrated in 20 mM Tris, 150 mM NaCl, 5 mM EDTA, pH 7.4. Following loading of the protein, the Chel x-100 column is washed with one column volume of the same buffer before disconnecting it from the series. The anion exchange column is washed with 3 column volumes of equilibration buffer before eluting the protein with 0.4 M NaCl, 20 mM Tris-acetate, pH 6.5. Protein concentrations of recombinant human protein C and recombinant activated protein C solutions are measured by UV 280 nm extinction E0.1%=1.81 or 1.85, respectively.

### Preparation 2

### Activation of Recombinant Human Protein C

Bovine thrombin is coupled to Activated CH-Sepharose 4B (Pharmacia) in the presence of 50 mM HEPES, pH 7.5 at 4° C. The coupling reaction is done on resin already packed into a 0.15 column using approximately 5000 units thrombin/ml resin. The thrombin solution is circulated through the column for approximately 3 hours before adding MEA to a concentration of 0.6 ml/l of circulating solution. The MEA-containing solution is circulated for an additional 10-12 hours to assure complete blockage of the unreacted amines on the resin. Following blocking, the thrombin-coupled resin is washed with 10 column volumes of 1 M NaCl, 20 mM Tris, pH 6.5 to remove all non-specifically bound protein, and is used in activation reactions after equilibrating in activation buffer.

Purified rHPC is made 5 mM in EDTA (to chelate any residual calcium) and diluted to a concentration of 2 mg/ml with 20 mM Tris, pH 7.4 or 20 mM Tris-acetate, pH 6.5. This material is passed through a thrombin column equilibrated at 37° C. with 50 mM NaCl and ither 20 mM Tris pH 7.4 or 20 mM Tris-acetate pH 6.5. The flow rate is adjusted to allow for approximately 20 min. of contact time between the rHPC and thrombin resin. The effluent is collected and immediately assayed for amidolytic activity. If the material did not have a specific activity (amidolytic) comparable to an established standard of aPC, it is recycled over the thrombin column to activate the rHPC to completion. This is followed by 1:1 dilution of the material with 20 mM buffer as above, with a pH of either 7.4 or 6.5 to keep the aPC at lower concentrations while it awaited the next processing step.

Removal of leached thrombin from the aPC material is accomplished by binding the aPC to an anion exchange resin (Fast Flow Q. Pharmacia) equilibrated in activation buffer (either 20 mM Tris, pH 7.4 or 20 mM Tris-acetate, pH 6.5) with 150 mM NaCl. Thrombin does not interact with the anion exchange resin under these conditions, but passes through the column into the sample application effluent. Once the aPC is loaded onto the column, a 2-6 column volume wash with 20 mM equilibration buffer is done before eluting the bound aPC with a step elution using 0.4 M NaCl in either 5 mM Tris-acetate, pH 6.5 or 20 mM Tris, pH 7.4. Higher volume washes of the column facilitated more complete removal of the dodecapeptide.

The anticoagulant activity of activated protein C was determined by measuring the prolongation of the clotting time in the activated partial thromboplastin time (APTT) clotting assay. A standard curve was prepared in dilution buffer (1 mg/mL radioimmunoassay grade bovine serum albumin [BSA], 20 mM Tris, pH 7.4, 150 mM NaCl, 0.02% NaN₃) ranging in protein C concentration from 125-1000 ng/mL, while samples were prepared at several dilutions in this concentration range. To each sample cuvette, 50 μL of cold horse plasma and 50 μL of reconstituted activated partial thromboplastin time reagent (APTT Reagent, Sigma) were added and incubated at 37° C. for 5 min. After incubation, 50 μL of the appropriate samples or standards were added to each cuvette. Dilution buffer was used in place of sample or standard to determine basal clotting time. The timer of the fibrometer (CoA Screener Hemostasis Analyzer, American Labor) was started immediately after the addition of 50 μL 37° C. 30 mM CaCl₂ to each sample or standard. Activated protein C concentration in samples are calculated from the linear regression equation of the standard curve. Clotting times reported here are the average of a minimum of three replicates, including standard curve samples.

### EXAMPLE 1

### Preparation of Des 416-419 Activated Protein C

aPC was used as the starting material to prepare des 416-419 aPC. Immobilized thrombin resin (10 mg thrombin/ml CH-Sepharose 4B resin) was used. N-glycosidase F was purchased from Roehringer Mannheim. Horse plasma is a product of Animal. Technologies, Inc. (Tyler, Tex.). Activated CH Sepharose® 4B was bought from Pharmacia Biotech. All other chemicals were ACS reagent grade and commercially available.

A 6 mL quantity of immobilized thrombin resin was put on a 0.2 micron filter. The resin was washed with approximately 5×20 mL of 40 mM tris buffer, pH 7.02. The washed immobilized thrombin resin was transferred to a 50 mL polypropylene vial, a 12 mL aliquot of a 2.67 mg/mL aPC solution (120 mg aPC in 45 mL of 40 mM tris buffer, pH 7.02) was added to the vial and the final volume of the suspension was adjusted to approximately 21 mL with tris buffer. The suspension was incubated at ambient temperature with constant gentle agitation. After incubation times of 10, 25, 50, 100, 160 and 240 min, 3 mL aliquots of the suspension were removed from the vial. These aliquots were centrifuged at 2000 RPM (ICE CRU-5000 Centrifuge) for 1 min. and the supernatants were transferred to several 1.5 mL polypropylene vials. These vials were immediately placed into a dry ice bath to freeze the solution. A control sample was prepared at the same time using de-activated CH-Sepharose 4B resin which did not contain immobilized thrombin.

Protein Content Assay. Aliquots,(150 mcL) of the sample solution was diluted with 450 mcL of 40 mM tris buffer, pH 7.02 or reagent water. The sample cell was rinsed twice with the sample solution and the UV absorbance (at λ=280 nm) of the solution was measured. Tris buffer or reagent water was used as the blank for this measurement.

LC/MS Assay for Protein Polypeptide Distribution. Aliquots of approximately 600 mcL of the sample solution were mixed with 240 mg urea, 88 mcL of 3 M tris buffer (pH=8.0) and 15 mcL of 50 mg/mL dithiothreitol solution and the mixture was incubated at 37° C. for 30 min. The sample was alkylated by adding 50 mcL of 50 mg/mL iodoacetamide solution and incubating at ambient temperature in the dark for 30 min. Samples were then desalted on a disposable gel filtration column, deglycosylated with N-glycosidase F and analyzed by LC/MS.

RP-HPLC Assay. Three hundred to four hundred microliter aliquots of the thawed sample solution were mixed with a sufficient volume of 0.1% TPA solution to obtain an approximately 1 mg/mL solution. This solution was used as the high concentration sample. The low concentration sample was prepared by mixing 50 mcL aliquots of the high concentration sample with 450 mcL of 0.1% TFA solution. One hundred microliter aliquots of each high and low concentration sample were injected onto the HPLC system.

APTT Assay. The sample was assayed on an Automated Activated Partial Thromboplastin Time (APTT) CoaLab Analyzer. All samples were diluted using manual pipettes to final concentrations between 410 ng and 420 ng aPC/mL. An aPC reference standard having an assigned potency of 303 U/mg, was used for this assay. Des (416-419) aPC generated as described above has similar biological activity to that of native aPC as measured by the APTT assay. The relationship between APTT anticoagulant activity and percent of Des 416-419 aPC is shown in Table 1. The percent of Des 416-419 aPC may be as high as 68% and still maintains essentially the same anticoagulant activity as native aPC.

In general, aPC made by the methods described herein contain from about it to about 25% Des 416-419 aPC.

TABLE 1

| | Percent (%) of Des | APTT Activity |
|---|---|---|
| Incubation | 416-419 aPC | (U/mg) |

| Time (min) | Control | Sample | Control | Sample |
|---|---|---|---|---|
| t = 0 | 13 | — | 512 | — |
| t = 10 | 14 | 20 | | 503 |
| t = 25 | 14 | 26 | | 533 |
| t = 50 | 14 | 35 | | 530 |
| t = 100 | 14 | 46 | | 521 |
| t = 160 | 14 | 57 | | 509 |
| t = 240 | 13 | 68 | | 509 |

What is claimed is:

1. An isolated human protein C polypeptide comprising:
a light chain and a truncated heavy chain.

2. The polypeptide of claim 1 wherein said polypeptide is SEQ ID NO: 1.

3. A recombinant DNA molecule encoding the human protein C polypeptide of claim 1.

4. The recombinant DNA molecule of claim 3, wherein said DNA molecule is SEQ ID NO: 2.

5. The isolated human protein C polypeptide of claim 1, wherein said human protein C polypeptide is activated.

6. A method of treating thrombotic disorders, vascular occlusive disorders and hypercoagulable states in a patient in need thereof, which comprises: administering to said patient a pharmaceutically effective amount of an isolated activated protein C polypeptide with a truncated heavy chain of claim 1.

7. A vector, comprising a nucleic acid according to claim 2.

8. A host cell comprising an isolated nucleic acid according to claim 2.


SEQUENCE LISTING (2 sequences)

SEQ ID NO: 1 - DNA, 1245 bp, Artificial Sequence
  Description of Artifical Sequence recombinant human protein c truncated at C-terminus
gccaactccttcctggaggagctccgtcacagcagcctggagcgggagtgcatagaggagatctgtgacttcgaggaggccaaggaaattttccaaaatgtggatgacacactggccttctggtccaagcacgtcgacggtgaccagtgcttggtcttgcccttggagcacccgtgcgccagcctgtgctgcgggcacggcacgtgcatcgacggcatcggcagcttcagctgcgactgccgcagcggctgggagggccgcttctgccagcgcgaggtgagcttcctcaattgctcgctggacaacggcggctgcacgcattactgcctagaggaggtgggctggcggcgctgtagctgtgcgcctggctacaagctgggggacgacctcctgcagtgtcaccccgcagtgaagttcccttgtgggaggccctggaagcggatggagaagaagcgcagtcacctgaaacgagacacagaagaccaagaagaccaagtagatccgcggctcattgatgggaagatgaccaggcggggagacagcccctggcaggtggtcctgctggactcaaagaagaagctggcctgcggggcagtgctcatccacccctcctgggtgctgacagcggcccactgcatggatgagtccaagaagctccttgtcaggcttggagagtatgacctgcggcgctgggagaagtgggagctggacctggacatcaaggaggtcttcgtccaccccaactacagcaagagcaccaccgacaatgacatcgcactgctgcacctggcccagcccgccaccctctcgcagaccatagtgcccatctgcctcccggacagcggccttgcagagcgcgagctcaatcaggccggccaggagaccctcgtgacgggctggggctaccacagcagccgagagaaggaggccaagagaaaccgcaccttcgtcctcaacttcatcaagattcccgtggtcccgcacaatgagtgcagcgaggtcatgagcaacatggtgtctgagaacatgctgtgtgcgggcatcctcggggaccggcaggatgcctgcgagggcgacagtggggggcccatggtcgcctccttccacggcacctggttcctggtgggcctggtgagctggggtgagggctgtgggctccttcacaactacggcgtttacaccaaagtcagccgctacctcgactggatccatgggcacatcagagacaaggaagccccccagaag

SEQ ID NO: 2 - protein, 415 aa, Artificial Sequence
  Description of Artifical Sequence recombinant human protein c truncated at C-terminus
ANSFLEELRHSSLERECIEEICDFEEAKEIFQNVDDTLAFWSKHVDGDQCLVLPLEHPCASLCCGHGTCIDGIGSFSCDCRSGWEGRFCQREVSFLNCSLDNGGCTHYCLEEVGWRRCSCAPGYKLGDDLLQCHPAVKFPCGRPWKRMEKKRSHLKRDTEDQEDQVDPRLIDGKMTRRGDSPWQVVLLDSKKKLACGAVLIHPSWVLTAAHCMDESKKLLVRLGEYDLRRWEKWELDLDIKEVFVHPNYSKSTTDNDIALLHLAQPATLSQTIVPICLPDSGLAERELNQAGQETLVTGWGYHSSREKEAKRNRTFVLNFIKIPVVPHNECSEVMSNMVSENMLCAGILGDRQDACEGDSGGPMVASFHGTWFLVGLVSWGEGCGLLHNYGVYTKVSRYLDWIHGHIRDKEAPQK